(19) United States
(12) Patent Application Publication
(10) Pub. No.: US 2007/0219464 A1
(43) Pub. Date: Sep. 20, 2007

(54) Guidewire with deflectable re-entry tip

(76) Inventors: Stephen Davis, Hopewell, NJ (US); David D. Grewe, Glen Gardner, NJ (US); Hikmat Hojeibane, Princeton, NJ (US); Steven Omiecinski, Princeton, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/590,537

(22) Filed: Oct. 31, 2006

Related U.S. Application Data
(63) Continuation-in-part of application No. 10/691,823, filed on Oct. 23, 2003.
Application No. 10/691,823 is a continuation-in-part of application No. 10/224,168, filed on Aug. 20, 2002, now Pat. No. 7,128,718.
(60) Provisional application No. 60/366,739, filed on Mar. 22, 2002.

(51) Int. Cl. A61M 25/01

(52) U.S. Cl. 600/585

(57) ABSTRACT

A steerable guidewire having a sharpened re-entry tip which comprises a longitudinal hypotube and an interlocking spring coil attached to the distal end of the hypotube and also includes a longitudinally movable deflection member which is attached to the distal end of the spring coil and a tip retaining member which extends from the distal end of the hypotube to the distal end of the spring coil for providing very precise deflection of the re-entry tip.

### CROSS-REFERENCE TO RELATED APPLICATION(S)

This patent application is a continuation-in-part of U.S. patent application Ser. No. 10/691,823 (Attorney Docket No. CRD1061USACIP1), filed on Oct. 23, 2003, entitled, “Guidewire With Deflectable Tip Having Improved Torque Characteristics,” which is a continuation-in-part of U.S. patent application Ser. No. 10/224,168 (Attorney Docket No. CRD1061USNP), filed on Aug. 20, 2002, entitled, “Guidewire With Deflectable Tip,” now issued as U.S. Pat. No. 7,128,718, which is a nonprovisional patent application of U.S. patent application Serial No. 60/366,739 (Attorney Docket No. CRD1035USPSP), filed on Mar. 22, 2002, entitled, “Deflection Wire Concept.”

### BACKGROUND OF THE INVENTION

1. Field of the Invention

The present invention relates to a steerable guidewire having a re-entry distal tip, and more particularly to a steerable guidewire having a sharpened tip which may be very precisely “steered,” and deflected. The guidewire is particularly suitable for use in providing a passageway around an obstruction within a blood vessel.

2. Description of the Prior Art

For many years guidewires have included a core wire with the distal end being tapered and with a coil spring mounted on the tapered distal end. These guidewires have been used to facilitate the insertion of a catheter into a vessel of the body. Generally, the guidewire is inserted into a vessel, a catheter is inserted over the guidewire and the catheter is then moved through the vessel until the distal end of the catheter is positioned at a desired location. The guidewire is then retracted from the catheter and the catheter is left in the vessel. Alternatively, the guidewire may be first inserted into the catheter with the distal portion of the guidewire extending beyond the distal end of the catheter. This assembly is then inserted into a vessel with the distal tip of the guidewire being used to facilitate movement of the guidewire and catheter through the vessel. Again, when the distal tip of the catheter has been placed in a desired location, the guidewire may be retracted thereby leaving the catheter in place within the vessel.

Another common application for guidewires is that of using the distal tip of the guidewire for removing an obstruction within a vessel. Often times this procedure is accomplished by inserting the guidewire within a vessel, moving the distal tip of the guidewire into contact with the obstruction and then very gently tapping the distal tip of the guidewire against the obstruction until the guidewire passes through the obstruction. Alternatively, various types of devices may be placed on the distal end of a guidewire for actively opening an obstruction within the vessel. Examples of such devices which may be placed on the end of the guidewires in order to open an obstruction are disclosed in the following U.S. Pat. Nos. to Robert C. Stevens: 5,116,350; 5,078,722; 4,936,845; 4,923,462; and, 4,854,325. Still another common application of guidewires is that of creating a passageway around the edge of an obstruction within a vessel, or alternatively, creating a passageway between the inner layer of a blood vessel, intimal, and the outer layer of the blood vessel.

While most guidewires used today do not include a mechanism for deflecting or steering the tip of the guidewire, it is very desirable to provide tip steering in order to facilitate movement of the guidewire through the tortuous vessels of the body, and then around an obstruction or through the wall of a vessel. There are many patents directed toward different mechanisms for deflecting the distal tip of a guidewire in order to steer the guidewire. Examples of such guidewires are disclosed in the following patents: U.S. Pat. No. 4,815,478 to Maurice Buchbinder, et al., U.S. Pat. No. 4,813,434 to Maurice Buchbinder, et al., U.S. Pat. No. 5,037,391 to Julius G. Hammerslag, et al., U.S. Pat. No. 5,203,772 to Gary R. Hammerslag, et al., U.S. Pat. No. 6,146,338 to Kenneth C. Gardeski, et al., U.S. Pat. No. 6,126,649 to Robert A. VanTassel, et al., U.S. Pat. No. 6,059,739 to James C. Baumann and U.S. Pat. No. 5,372,587 to Julius G. Hammerslag, et al. U.S. Pat. No. 4,940,062 to Hilary J. Hampton, et al., discloses a balloon catheter having a steerable tip section. All of the above-identified patents are incorporated herein by reference.

While each of the latter group of patents disclose guidewires having some degree of steerability, there is a need to have a guidewire with very precise steering in a guidewire of a very small diameter which is suitable for the purposes described above. More particularly, there is an important need for a very small diameter guidewire having improved torque characteristics which includes a sharpened distal tip which may be deflected very precisely to be directed within the vessel around an obstruction or through the wall of the vessel in order to provide a passageway.

### SUMMARY OF THE INVENTION

In accordance with one aspect of the present invention, there is provided a very small diameter steerable guidewire having a deflectable tip which includes an elongated flexible tubing, a flexible helical coil attached to the distal portion of the flexible tubing, an elongated deflection member which is slidably disposed within the tubing and within the helical coil. The flexible helical coil is formed from an elongated member having a rectangular, or square cross section, and having continuous undulations wherein the undulations of adjacent turns interlock with each other, i.e., peak undulation of one turn interlocking with valley undulation of adjacent turn, to thereby enhance the rotational rigidity, referred to as torque characteristic, of the coil. The proximal portion of the deflection member is of a cylindrical configuration and the distal portion is tapered to form a deflection member. Alternatively, the deflection member may take the form of a proximal cylindrical wire which is attached at its distal end to a deflection member. In addition, a retaining ribbon is attached to the distal end of the flexible tubing and is oriented to extend in a plane which is generally parallel to the plane of the ribbon portion of the deflection member. A sharpened needle is bonded to the distal end of the helical coil. The distal end of the deflection member and the distal end of the retaining ribbon are preferably bonded into a cavity which extends into the proximal face of the sharpened needle. Longitudinal movement of the deflection member causes the sharpened needle to be deflected. With the enhanced rotational rigidity of the coil portion or the guidewire, the entire guidewire has improved rotational rigidity.

In accordance with another aspect of the present invention, the continuous undulations take the form of a sinusoidal wave, or alternatively a square sinusoidal wave, having positive and negative peaks and in which the positive peaks of adjacent turns of coils engage negative peaks, or valleys, of adjacent turns.

In accordance with another aspect of the present invention, the retaining ribbon and the deflection member are preferably pre-shaped into a curved configuration to thereby cause the flexible helical coil to be biased into a normally curved shape.

In accordance with a further aspect of the present invention, the distal portion of the deflection member engages the cavity at the distal end of the needle at a location offset from the central axis of the needle, and the distal portion of the retaining ribbon engages the cavity at the distal end of the needle at a location offset from the center of the central axis of the needle and preferably in the opposite direction from the offset location of the deflection member.

In accordance with still another aspect of the present invention, the deflection member and the retaining ribbon are connected to each other within the attachment member. Preferably these two elements are formed as a single unitary element. In a preferred embodiment of the invention the cylindrical deflection member is flattened to form the deflection member and is further flattened at its distal end to form the retaining ribbon. The retaining ribbon is bent 180 degrees with respect to the deflection member to form a generally U-shaped bend to thereby establish a predetermined spacing between the ribbons and to also cause these ribbons to remain parallel to each other.

### BRIEF DESCRIPTION OF THE DRAWINGS

FIG. 1 is an enlarged elevational view of a balloon on a guidewire having a deflectable tip and control handle in accordance with the one aspect of the present invention;

FIG. 2 is an enlarged elevational sectional view showing the distal end of a guidewire in its normal pre-shaped position;

FIG. 2A is an expanded elevational view of the interlocking coil portion of the guidewire shown in FIG. 1.

FIG. 3 is an enlarged sectional view showing the distal end of the steerable guidewire of FIG. 2 rotated 90 degrees; and,

FIGS. 4 and 5 are sectional views showing the steerable guidewire deflected from its normal position to opposite extremes of deflection.

### DESCRIPTION OF THE PREFERRED EMBODIMENT

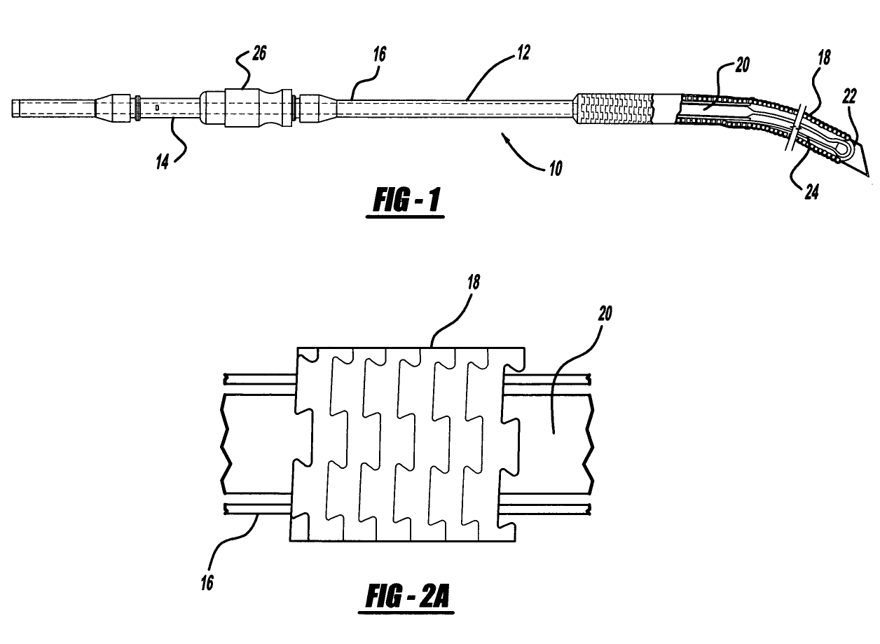

FIG. 1 generally illustrates a steerable guidewire system 10 which embodies the present invention and comprises a steerable guidewire 12 coupled to a control handle 14. More particularly, the steerable guidewire comprises an elongated hypotube 16, a helical coil 18 attached to and extending from the distal end of the hypotube 16. The helical coil 18 is of a rectangular or square cross-sectional configuration and is preferably formed from platinum tungsten with the proximal turns being wound such that adjacent turns of the proximal portion are in contact, or loosely interlocked with each other.

While the preferred embodiment of the present invention includes the helical coil 18, this element may take the form of any flexible rectangular or square cross-sectional member, such as for example a thin square metallic tube with or without portions of the tube removed, for example laser cutting, so as to form a very flexible cylindrical or square member. An elongated deflection member 20 extends from the proximal end of the control handle through the hypotube 16 and through the helical coil 18, and is connected to a beveled needle tip 22, which is also bonded to the distal tip of the helical coil 18. In addition, a retaining ribbon 24 is connected to the distal end of the hypotube 16 and is also connected to the needle tip 22 for controlling the deflection of the needle tip 22.

The control handle 14 generally comprises a slidable control knob 26 which may be moved longitudinally with respect to the control handle. The control handle 14 is coupled to the deflection member 20. As will be discussed in more detail, the longitudinal movement of the slidable control knob 26 causes deflection of the distal tip of the guidewire in either an upward or downward direction.

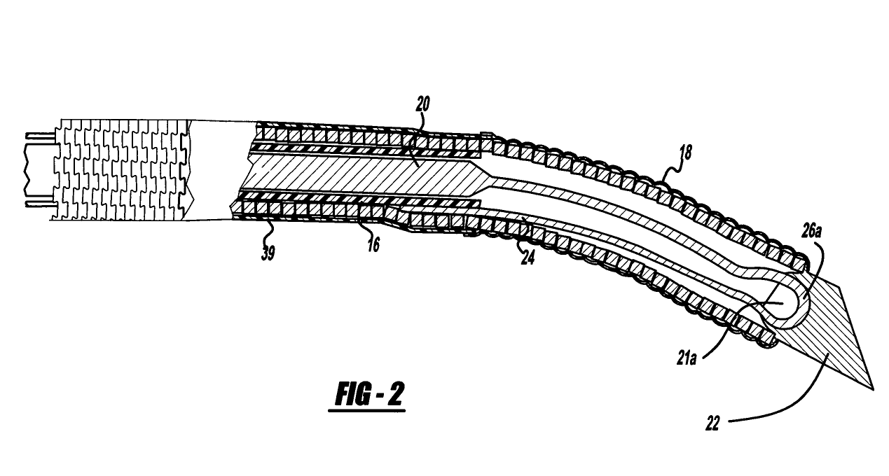

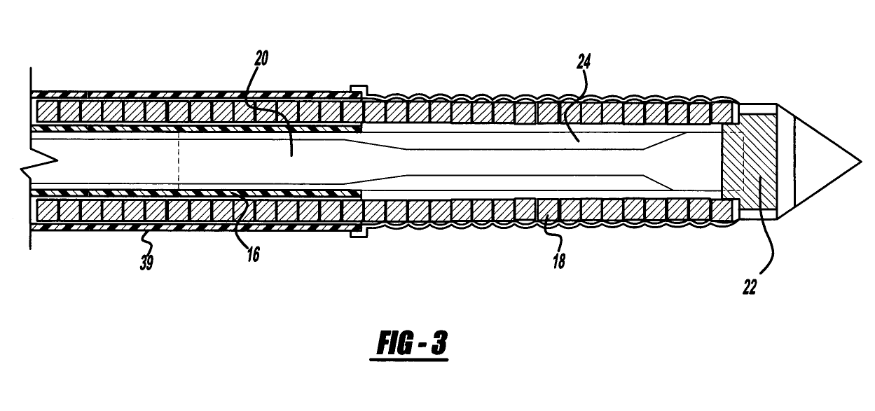

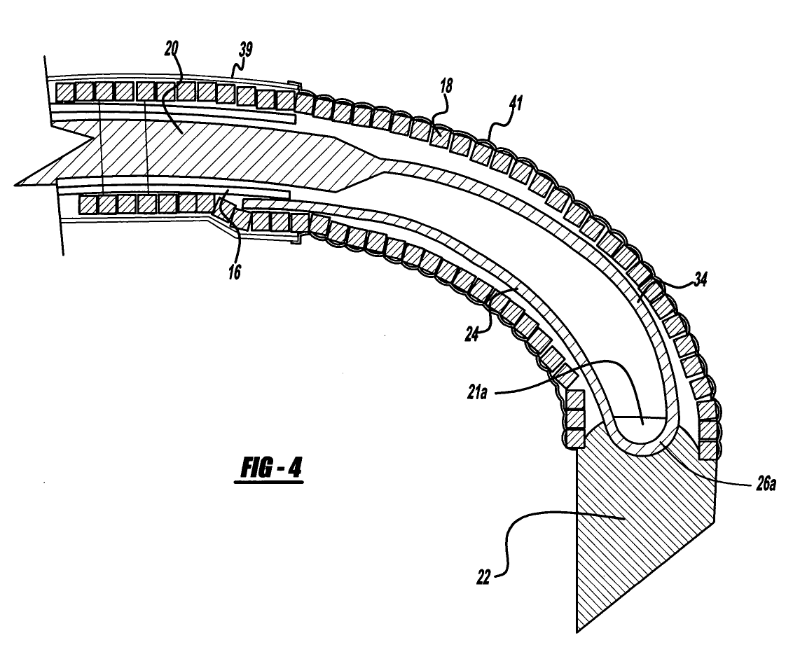

FIGS. 2, 2A and 3 illustrate in more detail the distal portion of the steerable guidewire 12 including the deflection mechanism and the needle tip 22. As may be appreciated, FIG. 3 is a view of the guidewire 12 shown in FIG. 2 with the guidewire being rotated 90 degrees about its longitudinal axis. More particularly, the proximal end of the helical coil 18 is bonded, preferably by use of an epoxy, to the outer surface near the distal end of the hypotube 16. The elongated deflection member 20 takes the form of a small diameter cylindrical wire having an intermediate portion which is flattened to form a thin ribbon having a thickness of approximately 0.002 inches. The distal end of the cylindrical deflection member 20 is further flattened to a thickness of approximately 0.0015 inches and is bent back 180 degrees to form a U-shaped bend 26a between the deflection member 20 and the retaining ribbon 24. The proximal end of the retaining ribbon 24 is bonded, preferably by use of epoxy, to the outer surface of the distal end of the hypotube 16. The retaining ribbon 24 is aligned in a plane parallel to the plane of the deflection member 20. The U-shaped portion between the ribbons is bonded, preferably by welding or use of an epoxy, into a cavity 21a formed within the distal portion of the needle tip 22.

The beveled needle tip 22 is preferably formed of platinum or iridium or other material which is radiopaque to X-rays so that the position of the needle tip may be clearly observed by fluoroscopy during an interventional procedure. The needle tip 22, as well as the guidewire 12 are of very small diameter so that the needle tip may either be steered around an obstruction in a blood vessel, i.e., between the obstruction and the wall of the vessel, or alternatively, between the inner layer intimal, of the blood vessel and the outer layer of the blood vessel. In either case a pathway may be provided around the obstruction.

As may be appreciated, with this unitary construction of the ribbon members, these members remain aligned so that both lie in planes parallel to each other. In addition, the U-shaped bend portion which is bonded to the needle tip 22 causes the retaining ribbon and deflection member to be properly spaced with respect to each other.

As illustrated in FIG. 2, the retaining ribbon 24 is preferably attached to the distal section of the needle tip 22 at a position offset from the longitudinal axis of the needle tip 22. In addition, the deflection member 20 is offset from the longitudinal axis of the needle tip 22 in an opposite direction from the offset of the retaining ribbon 24.

Also, as may be seen in FIG. 2, the deflection member 34 and the retaining ribbon 24 are pre-shaped into an arcuate, or curved, configuration to thereby maintain the helical coil 18 in a normally curved configuration. The deflection member 20 and the retaining ribbon 24 are pre-shaped such that the needle tip 22 curves away from the longitudinal axis of the guidewire in a direction toward that side of the guidewire containing the retaining ribbon 24, or downwardly as illustrated in FIG. 2.

The helical coil 18 is formed as an elongated member having a rectangular, or square, cross-sectional configuration and wound in a helical configuration. In addition as illustrated in FIG. 2A, the elongated member is formed with re-occurring steps, or step undulations, which when wound into a helical configuration so that adjacent turns to loosely interlock thereby preventing movement between adjacent turns. Such interlocking turns enhance the rotational rigidity or “torqueability” of the coil, such that when the proximal end of the coil is rotated 180 degrees, the distal end of the coil will rotate approximately 180 degrees. Accordingly, the distal end of the coil more nearly tracks, rotationally, the proximal end of the coil thereby significantly improving the “tortional” characteristics of the coil. By improving the “tortional” characteristics of the coil, the overall “tortional” characteristics of the guidewire are significantly improved.

As opposed to winding an elongated member to form the helical coil 18, a preferred method of forming the helical coil is by laser cutting the coil from a single thin-walled tube of an alloy in the undulations locking, stepped configuration as illustrated in FIG. 2A. Such laser cutting provides a coil with precise mating surfaces to assure proper interlocking between adjacent turns of the helical coil.

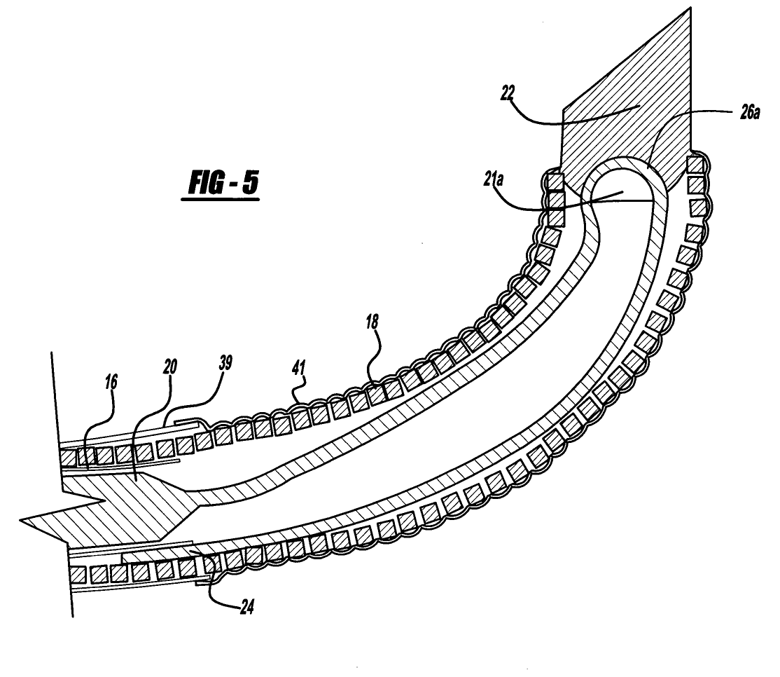

In operation, as previously described, the needle tip 22 is normally biased into a downwardly curved position as illustrated in FIG. 2 because of the curve of the pre-shaped deflection member 20 and the shortened length of the retaining ribbon 24. When the slidable control knob 26 is moved distally as shown in FIG. 4, the deflection member 20 will be moved distally thereby causing the deflection member 20 to move in a distal direction. As the deflection member is moved distally, a pushing force is applied to the top portion of the needle tip 22. Since the retaining ribbon 24 is attached to the lower portion of the needle tip 22 is maintained at a fixed distance from the distal end of the hypotube 16. As the deflection member 20 is moved to the right, the tip of the guidewire is caused to deflect downwardly to a maximum deflected position.

Since the deflection member 34 and the retaining ribbon 24 are pre-shaped prior to any activation of the steerable guidewire, the amount of force required to deflect the guidewire in the direction shown in FIG. 4 is very small thereby preventing buckling of the deflection member 20 as the deflection member is pushed distally. As the deflection member 20 is moved distally, the upper turns of the helical coil become slightly stretched and the lower turns of the coil become slightly compressed. The deflection member 20 has a diameter of about 0.0065 inches and the deflection member has a thickness of about 0.002 inches to thereby provide sufficient stiffness to prevent the buckling of these elements when the deflection member 20 is pushed distally. This construction also provides sufficient stiffness to transmit the necessary force from the proximal end to the distal end of the guidewire.

When the slidable control knob 26 is moved in a proximal direction as shown in FIG. 5, the deflection member 20 will be pulled to the left to thereby cause the deflection member 20 to pull on the top portion of the needle tip 22. Since again the retaining ribbon 24 causes the lower portion of the needle tip 22 to remain at a fixed distance from the distal end of the hypotube 16, the needle tip is caused to bend in an upward direction to a maximum deflection as shown in FIG. 4. Since the deflection member 20 is in tension when the deflection member is pulled, there is no concern for buckling of the deflection member. As the deflection member 20 is moved proximally, the upper coil turns become slightly compressed and the lower coil turns become somewhat stretched.

As previously discussed, when the proximal end of the guidewire 12 is rotated by a physician to “steer” the distal end of the guidewire, with the interlocking turns of adjacent coils of the helical coil 18, the distal tip will rotate on a one-to-one basis with respect to the proximal end of the hypotube 16. In other words, there is no “play” or “lag” between rotation of the proximal end and the distal end of the guidewire.

In a preferred embodiment of the present invention, the elongated deflection member 20 and the retaining ribbon 24 are constructed of nitinol, but these elements may be formed from other flexible materials including polymers. The helical coil 18 preferably formed by laser cutting as previously discussed, is constructed from an alloy comprised of about 92 percent platinum and 8 percent tungsten, but this element may also be constructed from numerous other materials. It is desirable that the coil exhibit the characteristic of being radiopaque to X-rays to assist in the positioning of the distal tip of the steerable guidewire 12. The deflection member 20 is formed from a single cylindrical nitinol wire of about 0.0065 inches in diameter having an intermediate portion which is flattened to form the deflection member 20 with a thickness of about 0.002 inches, and a distal portion which is flattened to form the retaining ribbon 24 with a thickness of about 0.0015 inches. The retaining ribbon 24 is bent back 180 degrees to form a generally U-shaped bend, which is subsequently bonded into the proximal cavity 21a of the needle tip 22.

It has been found that the addition of graphite between the deflection member 20 and deflection member 34, and the inner lumen of the hypotube 16 provides lubrication. Other lubricants, such as Teflon or MDX may be used for this purpose. The helical coil 18 is preferably coated with an elastomeric polymer 41 on its distal end to act as a sealant preventing the entry of blood and contrast media into the guidewire and a fluorinated polymer 39, such as Teflon, on its proximal end for lubrication purposes.

It may be seen that the guidewire as disclosed may be very easily and very precisely rotated and then very precisely deflected in either of two directions for very exacting steering of the guidewire through the vessels of the body. Accordingly, the disclosed guidewire may be used to cross an obstruction within a vessel or it may be used to create a passageway through the space between the inner layer, or intimal, and the outer layer of a blood vessel. To accomplish this latter procedure, the needle tip 22 is deflected so that it points toward the vessel wall and is advanced through the intimal layer and into the space between the inner layer and the outer layer of the vessel. After the needle tip 22 enters this space it may be moved beyond the location of the obstruction and then may be “steered” back through the intimal layer and into the lumen of the vessel to thereby provide a pathway around the obstruction.

The preceding specific embodiment is illustrated of the practice of this invention. It is to be understood, however, that other variations may also be employed, such as having the needle tip 22 being formed with a conical point or with a cutting configuration, without departing from the spirit and scope of the invention as hereinafter claimed.

That which is claimed is:

1. A steerable guidewire having a re-entry needle tip which comprises:
an elongated flexible tubing having proximal and distal portions;
a flexible helical coil having multiple turns and having proximal and distal ends;
an elongated deflection member having proximal and distal portions and being slidably disposed within said tubing and within said helical coil, the distal portion of said deflection member being flattened to form a deflection member which extends in a plane;
a retaining ribbon having proximal and distal ends, the proximal end of the retaining ribbon is attached to the distal portion of the flexible tubing and the retaining ribbon is oriented to extend in a plane which is generally parallel to the plane of the distal portion of the deflection member;
a sharpened needle tip is disposed at the distal end of the helical coil; and,
the distal portion of the deflection member and the distal end of the retaining ribbon are bonded to the proximal end of the needle tip so that longitudinal movement of the deflection member in a distal direction causes the needle tip to be deflected in one direction and longitudinal movement of the deflection member in a proximal direction causes the needle tip to be deflected in an opposite direction.

2. A steerable guidewire as defined in claim 1, wherein the needle tip is beveled at its distal tip to form a sharpened point.

3. A steerable guidewire as defined in claim 2, wherein the needle tip has a cavity disposed within the proximal end of the needle tip and the deflection member and retaining ribbon are bonded into said cavity.

4. A steerable guidewire as defined in claim 1, wherein the helical coil is formed from a spiral wound elongated member having continuous undulations and wherein the continuous undulations take the form of a sinusoidal wave having positive and negative peaks and in which the positive peaks of adjacent turns of coils engage negative peaks of adjacent turns.

5. A steerable guidewire as defined in claim 4, wherein the continuous undulations take the form of a square sinusoidal wave having positive and negative peaks and in which the positive peaks of adjacent turns of coils engage negative peaks of adjacent turns.

6. A steerable guidewire as defined in claim 1, wherein the elongated deflection member has a square cross-sectional configuration.

7. A steerable guidewire as defined in claim 1, wherein the retaining ribbon and the deflection member are normally biased in an arcuate configuration to thereby cause the distal end of the helical coil to be normally biased in a curved shape.

8. A steerable guidewire as defined in claim 1, wherein the proximal portion of said deflection member is of a circular cross section which extends from the proximal portion of the flexible tubing to approximately the distal portion of the tubing.

9. A steerable guidewire as defined in claim 8, wherein the proximal end of said retaining ribbon extends from the distal portion of the flexible tubing to approximately the distal end of the flexible helical coil.

10. A steerable guidewire as defined in claim 9, wherein the distal end of the retaining ribbon engages the needle tip at a location offset from a longitudinal axis of the needle tip.

11. A steerable guidewire as defined in claim 10, wherein the distal end of the retaining ribbon engages the needle tip at a location offset from the longitudinal axis of the needle tip in an opposite direction from the location of the deflection member.

12. A steerable guidewire as defined in claim 11, wherein the deflection member and the retaining ribbon are joined to each other within a cavity disposed within the proximal end of the needle tip.

13. A steerable guidewire as defined in claim 12, wherein the deflection member and the retaining ribbon are formed as a single unitary element.

14. A steerable guidewire as defined in claim 13, wherein the deflection member and the retaining ribbon are joined to form a generally U-shaped configuration to thereby provide a predetermined spacing between the deflection member and the retaining ribbon and to maintain the deflection member and the retaining ribbon in planes which are parallel to each other.

15. A steerable guidewire as defined in claim 14, wherein the deflection member is formed by flattening an intermediate portion of the deflection member, and the retaining ribbon is formed by flattening a distal portion of the deflection member.

16. A steerable guidewire as defined in claim 15, wherein the retaining ribbon is of a thickness which is less than the thickness of the deflection member.

17. A steerable guidewire as defined in claim 16, wherein the deflection member is of a thickness equal to about 0.002 inches and the retaining ribbon is of a thickness equal to about 0.0015 inches.
